(19) United States
(12) Patent Application Publication
(10) Pub. No.: US 2022/0293278 A1
(43) Pub. Date: Sep. 15, 2022

(54) CONNECTED CONTACT TRACING

(71) Applicant: Alarm.com Incorporated, Tysons, VA (US)

(72) Inventors: Matthew Daniel Correnti, Newtown Square, PA (US); Robert Nathan Picardi, Herndon, VA (US); Donald Gerard Madden, Columbia, MD (US); Michael Kelly, Washington, DC (US)

(21) Appl. No.: 17/674,023

(22) Filed: Feb. 17, 2022

Related U.S. Application Data
(60) Provisional application No. 63/160,032, filed on Mar. 12, 2021.

(51) Int. Cl. G16H 50/30; G16H 50/80; H04L 51/046; G06V 20/52; G06V 40/20; G06V 40/16

(52) U.S. Cl. CPC G16H 50/30; G16H 50/80; H04L 51/046; G06V 20/52; G06V 40/20; G06V 40/172

(57) ABSTRACT

Methods, systems, and apparatus, including computer programs encoded on a computer storage medium, for connected contact tracing system. The methods, systems, and apparatus include actions of receiving sensor data from multiple sensors, receiving exposure information including a person and an epidemiological event data, determining, from the sensor data, a contact exposure event including the person and another person, generating, from the sensor data and the exposure information, a risk score for the contact exposure event, and providing a notification including the risk score and information for the contact exposure event.

### CROSS-REFERENCE TO RELATED APPLICATIONS

This application claims the benefit of U.S. Provisional Application No. 63/160,032, filed Mar. 12, 2021, which is incorporated by reference in its entirety.

### TECHNICAL FIELD

This disclosure relates generally to contact tracing.

### BACKGROUND

Contact tracing can assist in curtailing spread of disease by identifying people who have had potential exposure to an infected person and alerting affected people of the risk of exposure. Collection and analysis of exposure data can assist in further mitigating the spread of the disease, provide exposed individuals with potential diagnosis and treatment options, and study the epidemiology of the disease.

### SUMMARY

Techniques are described for a connected contact tracing system.

More specifically, techniques are described for a contact tracing system that receives multiple streams of sensor data from multiple distributed sensors located around a building, e.g., a home or commercial building, and determines a risk score for each person who was potentially exposed to an infected person. Determining the risk score can include validating a potential exposure using multiple different streams of sensor data from multiple different sensors, e.g., video data and connectivity data from a wireless network. Determining the risk score can additionally incorporate environmental factors, e.g., state of an HVAC system, mitigation steps, e.g., presence of barriers, masking, antimicrobial measures, etc., and epidemiological information, e.g., modes of transmission. Various risk factors can be identified based on the sensor data, e.g., proximity and/or duration of exposure, spreading behaviors (e.g., coughing, sneezing, high touch surfaces), sharing of common objects, and the like. Different weights can be applied to the various risk factors, e.g., based on modes of transmission, to generate a risk score for each individual.

An alert including the risk score can be provided to each potentially exposed individual. Alerts can include details contextualizing the risk score, associated risk factors, as well as provide epidemiological information related to the exposure, e.g., symptoms, treatment, and mitigation strategies to prevent further spread.

Implementations of the described techniques may include hardware, a method or process implemented at least partially in hardware, or a computer-readable storage medium encoded with executable instructions that, when executed by a processor, perform operations.

The techniques described in this disclosure provide one or more of the following advantages. Utilizing a combination of multiple sensor data streams can result in a more accurate and/or more informative contact tracing, e.g., by incorporating environmental factors, mitigation steps, etc., when calculating a risk score for an exposed person. The contact tracing system can be flexible to account for modes of transmission/risk factors for a particular epidemiological event, such that risk score calculations more accurately reflect real risk to exposed persons. Utilizing multiple data streams, e.g., video data and connectivity data, can further pinpoint physical/temporal overlap of a potentially exposed person with an infected person and determine potential modes of exposure between the infected person and the potentially exposed person, and can lead to an increased confidence about potential exposure.

In some embodiments, associations can be determined between exposure risk and specifics areas/factors of the property environment, where additional mitigation steps can be taken in response to reduce risk in a targeted manner.

In some aspects, the subject matter described in this specification may be embodied in methods that may include the actions of receiving sensor data from multiple sensors, receiving exposure information including a person and an epidemiological event data, determining, from the sensor data, a contact exposure event including the person and another person, generating, from the sensor data and the exposure information, a risk score for the contact exposure event, and providing a notification including the risk score and information for the contact exposure event.

Other versions include corresponding systems, apparatus, and computer programs, configured to perform the actions of the methods, encoded on computer storage devices.

These and other versions may each optionally include one or more of the following features. For instance, in some implementations determining, from the sensor data, a contact exposure event including the person and another person includes identifying, based on the sensor data, presence of the person at a property, identifying, based on the sensor data, presence of the another person at the property, and determining the contact exposure event based on the presence of the person at the property and the presence of the another person at the property.

In certain aspects, identifying, based on the sensor data, presence of the person at a property includes identifying the person at the property based on imaging data from a camera system installed in the property. In some aspects, generating, from the sensor data and the exposure information, a risk score for the contact exposure event includes determining a duration and a proximity of the person and the another person at a property based on the sensor data and generating the risk score for the contact exposure event based on the duration and the proximity of the person and the another person at the property based on the sensor data.

In some aspects, generating the risk score for the contact exposure event is based on sensor data from a heating, ventilation, and air conditioning system installed in a property. In some implementations, generating, from the sensor data and the exposure information, a risk score for the contact exposure event includes determining, based on the sensor data, that the person was sneezing or coughing at a property and generating the risk score for the contact exposure event based on that the person was sneezing or coughing at a property.

In certain aspects, receiving exposure information including a person and an epidemiological event data includes receiving an indication that the person was infected with an illness at a particular time. In some aspects, receiving an indication that the person was infected with an illness at a particular time includes receiving an indication that the person that was infected with an illness visited a particular subarea in a property at a particular time.

The details of one or more implementations are set forth in the accompanying drawings and the description below. Other features will be apparent from the description and drawings, and from the claims.

### BRIEF DESCRIPTION OF THE DRAWINGS

FIG. 1 is an example operating environment for a contact tracing system.

FIG. 2 is a flow diagram of an example process of a contact tracing system.

FIG. 3 shows a diagram illustrating an example home monitoring system.

Like reference symbols in the various drawings indicate like elements.

### DETAILED DESCRIPTION

Techniques are described for a connected contact tracing system including a comprehensive network of distributed sensors generating multiple data streams to identify, track, and mitigate exposures to infected individuals.

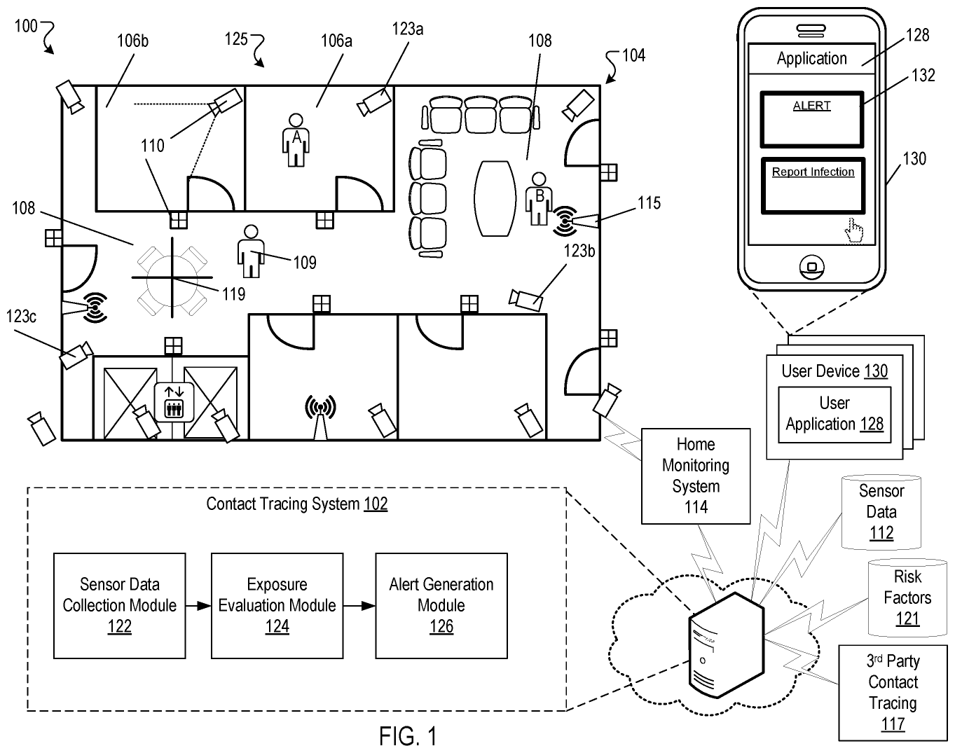

FIG. 1 is an example operating environment 100 for a contact tracing system 102. A property 104, e.g., a residence, multi-dwelling unit, commercial building, government building, etc., can include multiple sub-areas 106a, 106b. Each sub-area 106a can be a separate residence or commercial space, e.g., a different apartment, townhouse, business, etc., that shares a common area 108, e.g., shared hallways, staircases, lobby, entrances/exits, etc. Each residence or commercial space of the property 104 can be further divided into respective sub-areas, e.g., rooms within an apartment, or office spaces. Sub-areas 106a, 106b can each have a respective smart home or commercial system including a hub, e.g., a home monitoring system 114, where the respective home monitoring systems 114 from each sub-area 106a,b can be connected to a same service provider. Sensor data 112 that is collected, e.g., by sensors, smart appliances, user devices, etc., by each home monitoring system 114 can be shared over a network 116 to a centralized service provider which may utilize the collected sensor data 112 to perform contact tracing.

Sub-areas 106a, 106b and common area 108 can include multiple sensors 110 that each collect respective sensor data 112 representative of a state of the sub-area 106a, 106b in which the particular sensor 110 is located. Sensors 110 can include cameras, door locks, contact sensors, RFID badge readers, internet-of-things (IoT)-enabled smart appliances, passive infrared (PIR) motion sensors, radio detection and ranging (RADAR) systems/devices, light detection and ranging (LIDAR) systems/devices, time-of-flight or other similar depth sensors, microphones/audio receivers, seismic sensors, or the like. Each sensor 110 can generate respective sensor data 112, e.g., imaging data for a camera. Sensors 110 can be in data communication with a home monitoring system 114 and the contact tracing system 102 via a network 116. Network 116 can include one or more servers 118 that can host the home monitoring system 114 and contact tracing system 102.

In some implementations, sensors 110 include environmental sensors that record environment factors/conditions of the property 104. Environmental sensors can collect sensor data 112 related to temperature, humidity, airflow/circulation, ambient light levels, HVAC operation, filter operation, and the like. One or more of the environmental sensors can be a sub-component of an HVAC system of the property 104 and/or a sub-component of a smart appliance (e.g., a smart light bulb, a smart air purifier, etc.). Contact tracing system 102 can utilize sensor data 112 collected from environmental sensors to determine environmental factors for the property 104, for example, a local temperature and/or humidity level of a sub-area 106a or common area 108.

In some implementations, property 104 includes wireless connectivity devices 115, e.g., Wi-Fi hot-spots, Bluetooth (BT) low energy devices, etc., distributed around the property 104 and that collect connectivity data for user devices in data communication with the wireless connectivity device 115. User devices can include, for example, mobile phones, tablets, laptop computers, smart devices (e.g., smart watches), and the like. Connectivity data, e.g., strength of signal, collected by the wireless connectivity devices 115 and a device can be utilized to track a user device as it is moved through the property 104. For example, connectivity data between a user device and multiple wireless connectivity devices 115 distributed around the property 104 can be utilized to determine a geo-path of a user of the user device. In one example, the contact tracing system 102 can determine locations and duration of a person within property 104 based in part on connectivity data generated between the person's mobile phone and wireless connectivity device 115.

In some implementations, a wireless connectivity device 115 is a Bluetooth low energy device, e.g., a Bluetooth (BT) beacon. A BT beacon can be a sub-component of a user device, e.g., a mobile phone, where phone-to-phone Bluetooth low energy (BLE) detection can be performed between mobile phones. Sensor data 112 can include BLE detection between user devices belonging to various users, where users of the user devices have opted into sharing the collected data with the contact tracing system 102. In one example, sensor data 112 includes BLE detection between a first BT beacon in an infected person 109's mobile phone and each other mobile phone including a BT beacon with which the first BT beacon exchanges BLE signal.

In some implementations, sensor 110 is a global positioning system (GPS)-enabled device that is a sub-component of a user device, e.g., a GPS on a mobile phone. The GPS-enabled device can generate sensor data 112 including geolocation information, where the user may opt into sharing the collected geolocation information with the contact tracing system 102. For example, a user's mobile phone includes a GPS and shared location data with the contact tracing system 102. In some implementations, geolocation information for a user can be inferred from locations of the system's sensors and/or beacons. For example, the system 102 can infer a location of a user based in part on data collected from triggering of particular motion sensors, badge readers, door locks, etc. In another example, the system 102 can infer a location of a user based on connectivity data collected from one or more beacons, which is described in further detail below.

Network 116 can be configured to enable exchange of electronic communication between devices connected to the network 116. The network 116 can include, for example, one or more of the Internet, Wide Area Networks (WANs), Local Area Networks (LANs), analog or digital wired and wireless telephone networks (e.g., a public switched telephone network (PSTN), Integrated Services Digital Network (ISDN), a cellular network, and Digital Subscriber Line (DSL), radio, television, cable, satellite, or any other delivery or tunneling mechanism for carrying data. Network 116 may include multiple networks or subnetworks, each of which may include, for example, a wired or wireless data pathway. Network 116 may include a circuit-switched network, a packet-switched data network, or any other network able to carry electronic communications (e.g., data or voice communications). For example, network 116 may include networks based on the Internet protocol (IP), asynchronous transfer mode (ATM), the PSTN, packet-switched networks based on IP, X.25, or Frame Relay, or other comparable technologies and may support voice using, for example, VoIP, or other comparable protocols used for voice communications. Network 116 may include one or more networks that include wireless data channels and wireless voice channels. Network 116 may be a wireless network, a broadband network, or a combination of networks includes a wireless network and a broadband network.

In some implementations, sensors 110 include a camera system 125. Camera system 125 includes multiple cameras 123a-c, where each camera captures at least a portion of a sub-area 106a, 106b and/or common area 108 within a field of view of the camera. Imaging data captured by camera system 125 can be provided to the contact tracing system 102 over the network 116. Imaging data can be pre-processed by the camera system, for example, to identify objects/humans of interest within the collected imaging data using image processing software and video analytics.

Contact tracing system 102 includes sensor data collection module 122, exposure evaluation module 124, and alert generation module 126. Though described herein with reference to sensor data collection module 122, exposure evaluation module 124, and alert generation module 126, the actions can be performed by more or fewer modules. The sensor data collection module 122 can receive sensor data 112 from multiple different sensors 110 associated with the property 104.

Sensor data collection module 122 can receive, from multiple sensors 110, sensor data 112 as input. Sensor data 112 can be requested by the sensor data collection module 122 and/or a sensor 110 can push sensor data 112 to the sensor data collection module, for example, at periodic intervals. For example, sensor data collection module 122 can request updated sensor data 112 from the sensor 110 at a periodic interval, e.g., every 15 minutes, every 5 minutes, every hour, etc. In another example, the sensor 110 can provide updated sensor data 112 to the sensor data collection module 122 at a periodic interval, e.g., every 10 minutes, every 30 minutes, etc.

In some implementations, sensors 110 can provide sensor data 112 in response to determining an occurrence of an exposure event 113, e.g., detecting a person at the property 104. For example, a camera 123a can detect the presence of a person walking through area 108 and provide sensor data 112 including the detection to the sensor data collection module 122.

The sensor data collection module 122 can aggregate the sensor data 112 from multiple sensors 110 including metadata for the respective sensor data 112, e.g., time/date of the data, the particular sensor 110 that generated the sensor data, location of the particular sensor. The aggregated sensor data can be provided by the sensor data collection module 112 as output to the exposure evaluation module 124.

In some implementations, contact tracing system 102 receives a notification including information related to an infected person 109. The notification can be self-reported by the infected person 109 and/or can be provided by a 3^{rd} party contact tracing entity 117, e.g., a public health or government entity.

In some implementations, an infected person 109 may self-report an infection through an application 128 on a user device 130. Application 128, e.g., a contact tracing application, can include a graphical user interface through which a user may interact with the contact tracing application. Application 128 can serve as an interface between users of property 104, e.g., residents, visitors, employees, etc., and the contact tracing system 102. The infected person 109 may provide self-reported details of the infection, e.g., diagnosis, symptoms, onset of symptoms, etc. Additionally, the infected person 109 may self-report previous presence at the property 104, as well as details of locations visited within the property 104. In one example, an employee who has contracted influenza may self-report through application 128 a date/time of onset of symptoms and details related to sub-areas 106a,b and common area 108 last visited.

In some implementations, contact tracing system 102 can perform contact tracing among multiple locations, e.g., multiple properties 104. Contact tracing system 102 can receive aggregated sensor data 112 and information related to infected person 109s from multiple locations. 3rd party contact tracing entity 117 can provide contact tracing information related to infected person 109 amongst multiple properties at different locations to the contact tracing system 102.

The exposure evaluation module 124 can receive information related to an infected person 109 associated with the property 104. The information can include details of the infected person 109's presence at the property, e.g., time/date of visit, duration of visit, locations visited within the property, etc. The information can include details related to the infected person 109, e.g., a photo of the person, badge credentials, etc. The exposure evaluation module 124 can receive the information related to the infected person 109 and request aggregated sensor data 112 from multiple sensors 110 from sensor data collection module 122.

The exposure evaluation module 124 can receive the aggregated sensor data 112 and information related to the infected person 109 as input, and determine a risk score based on various exposure events identified in the sensor data 112 and risk factors 121 to generate an overall risk score for each potentially exposed person A and B who came in contact with the infected person 109 as output. Various factors can be included in a determination of the overall risk score, with some factors resulting in a lower risk score and others resulting in a higher risk score, as discussed in further detail below.

Determining a risk score can include utilizing data analytics, e.g., image processing, object/human recognition, etc., to identify persons who came into direct and/or indirect contact with the infected person 109 for a time period including when the infected person 109 was potentially contagious, e.g., 48 hours prior to showing symptoms. In one example, the exposure evaluation module 124 can generate a risk score for a person who shared an elevator with the infected person 109 that reflects a likelihood that the person was exposed to the infection of the infected person 109, e.g., contracted the infection.

The exposure evaluation module 124 can determine a risk score, based on various inter-related factors including, for example, proximity, duration, mitigating factors, environmental factors, and modes of transmission of the infection. A risk associated with each of the factors may be affected by one or more other of the factors, for example, a risk associated with a proximity between the infected person and another person can be reduced if one or more of the people involved are wearing a mask (and the infection is spread through aerosolized droplets).

In some implementations, the exposure evaluation module 124 can determine a risk score from the aggregated sensor data 112, based on a proximity of exposure of each person at the property 104, e.g., person A and person B, to the infected person 109. In some implementations, proximity of exposure can be determined, for example, using video analytics, phone to phone BLE detection, GPS location data of each potentially exposed person A, B and the infected person 109, access control data (e.g., from badge sensors, elevator codes, etc.). In one example, exposure evaluation module 124 can utilize facial recognition software to process video data from camera system 125 to identify and track the infected person 109 and each of the potentially exposed people through property 104. The contact tracing system 102 may build human models for each person identified in video data collected from camera system 125 to track movement of people between the various cameras 123a,b of the camera system 125. The contact tracing system 102 can determine, based on the aggregated sensor data 112, that a person A was within a threshold proximity of the infected person (e.g., within 6 feet of the infected person), and determine, based on this determined proximity, a risk score for person A. In one example, a closer proximity of person A with the infected person 109 results in a higher risk score and a farther proximity of person A with the infected person 109 results in a lower risk score.

In some implementations, the exposure evaluation module 124 can determine a risk score based on a duration of exposure of each person at the property 104 to the infected person 109. Duration of exposure can be determined, for example, by video analytics (e.g., observing the duration of proximity), phone to phone BLE detection (e.g., how long was there at least a threshold amount of signal exchange), access control data (e.g., two people rode an elevator together based on key card access), and the like. The contact tracing system 102 can determine, based on the aggregated sensor data 112, that a person A was within a threshold proximity of the infected person (e.g., within 6 feet of the infected person) for a threshold duration, and determine, based on this determined proximity and duration, a risk score for person A. In one example, video analytics and access control data can be utilized to determine how long person A was on a shared elevator ride with the infected person 109, e.g., which floors each person got on/off the elevator to determine an overlap time. A lower risk score can be assessed for person A for a duration of proximity that is less than a threshold duration, and a higher risk score can be assessed for a person A for a duration of proximity that is greater than a threshold duration.

In some implementations, the exposure evaluation module 124 can determine a risk score based on spreading behaviors performed by the infected person 109. For example, the exposure evaluation module 124 can apply video analytics (e.g., gesture recognition software) on video data collected from camera system 125 to determine if the infected person 109 performed spreading behaviors, e.g., coughing, talking, laughing, sneezing. Video analytics can be utilized to identify and categorize gestures into low risk or high risk categories, identify affected surfaces (e.g., door knobs contacted after a sneeze), etc. For example, high risk behaviors can include sneezing/coughing within a threshold proximity of another person, walking into an area where a person has previously sneezed/coughed within a threshold amount of time, etc. In another example, low risk behaviors can include sitting in a same common area a safe distance apart (e.g., more than 6 feet apart) while wearing protective equipment (e.g., masks). Video analytics, e.g., gesture recognition, facial recognition, objection recognition, etc., can be utilized to identify a person and a behavior.

In some implementations, the exposure evaluation module 124 can determine a risk score based on indirect contact between infected person 109 and potentially exposed persons A, B, e.g., through shared objects and/or locations. For example, the exposure evaluation module 124 can apply video analytics (e.g., facial recognition software, object recognition software) to identify common objects, surfaces, shared chair/workspaces, etc. In some implementations, the exposure evaluation module 124 can analyze access control, e.g., authentication data on shared terminals, to identify people who used a terminal (e.g., computer, ATM, vending machine, elevator panel, etc.) within a threshold amount of time after the infected person 109. Indirect contact can result in a higher or lower risk score, for example, touching an ATM directly after an infected person can result in an increased risk score. Indirect contact can increase/decrease risk score depending in part on modes of transmission of the infection, e.g., for an infection that has low spread on surfaces, the risk score may be low for indirect contact.

In some implementations, the exposure evaluation module 124 can determine a risk score based on mitigating environmental factors, e.g., presence of shields, masks worn by people, use of disinfectant/cleaning, etc. In one example, the exposure evaluation module 124 can utilize video analytics to identify these mitigating environmental factors, e.g., to identify masks, shields, cleaning staff, etc. Environmental factors may increase or decrease a risk score for a person A, for example, the presence of a shield 119 between person A and an infected person 109 can reduce a risk score for person A. In another example, if a common surface is not disinfected between use by an infected person 109 and person A, then a risk score can be increased.

In some implementations, a user may provide information related to environmental factors, e.g., provide information about locations and specifications of environmental factors. For example, a user may provide locations and dimensions of shields located in common area 108 to the system 102. In another example, a user may provide a cleaning/disinfecting schedule/regiment to the system 102 (e.g., when/where cleaning staff is present).

In some implementations, home monitoring system 114 can provide operational data from appliances, sub-systems, etc., connected to the home monitoring system 114 to the contact tracing system 102 to determine environmental factors. For example, home monitoring system 114 can collect operational data from an HVAC system (e.g., on/off cycles, filter status, temperature/humidity) and provide the collected data to the contact tracing system 102. In another example, home monitoring system 114 can collect operational data from smart appliances (e.g., on/off, programs run, operators using the appliance, etc.) and provide the collected data to the contact tracing system 102.

In some implementations, the exposure evaluation module 124 can determine a risk score based on environmental factors, e.g., a state of the HVAC system, temperature, humidity, windows open/closed, etc. In one example, the exposure evaluation module 124 can utilize sensor data 112 collected from an HVAC system to identify a state of the air circulation (e.g., system on vs. off, quality of the filter, etc.) within a sub-area 106a. Environmental factors can each raise or lower a risk of score, e.g., open windows, increased ventilation can result in a lowering of the risk score. In one example, an HVAC system that is actively circulating air through a HEPA filter can result in a lowered risk score.

In some implementations, the exposure evaluation module 124 can determine a risk score based on a combination of two or more of the proximity of contact, duration of contact, spreading behaviors, environmental factors, mitigating factors, modes of transmission, and indirect contact.

In some implementations, the exposure evaluation module 124 can utilize a risk score model to determine a risk score, where the risk score model can differ for different pathogens or categories of pathogens. For example, for certain airborne pathogen, the risk score model can weight risks and mitigating factors related to airborne transmission higher and weight risks and mitigating factors related to surface transmission and/or food-borne transmission lower.

For example, for a possible elevator contact exposure scenario, an infected person A rides an elevator for 90 seconds and then exits, and then a second person B enters the elevator 30 seconds later. For an example of an airborne pathogen, the risk score model may factor in, for example, 1) how much pathogen can be output into the elevator by person A (e.g., how long their ride was in the elevator, how much they were coughing, what surfaces they touches, what PPE if any was used), 2) the likelihood that the environment (e.g., air and surfaces) would contain significant levels of the pathogen (e.g., based on a particular pathogen, air turnover, filtration, amount of time passed, types of surfaces, amount of sunlight, etc.), and 3) an amount of exposure person B can experience based on the factors observed.

In some implementations, a simple weighting can be utilized for a risk score model. The risk score model can include a complex relationship between factors, modes of transmission, and mitigation factors. A risk score model can account for multiple modes of transmission (e.g., surface and airborne) and incorporate each risk and exposure level to the resulting risk score. For example, a risk score model can account for two paths for infection (e.g., air and surface) such that a total risk score can be calculated as: (weight of airborne risk)*(weighted sum of airborne risks)*(1−weighted sum of airborne mitigations)+(weight of surface risk)*(weighted sum of surface risks)*(1−weighted sum of surface mitigations).

In some implementations, multiple streams of data can be compared to enrich a determination of actual risk for an exposure event. For example, Bluetooth proximity could indicate that person A kept a safe distance from others within the office, and thus had no risk of exposure. However, video analytics of video data may indicate that person B touched the same elevator button as an infected person 109, which can indicate a high risk and thus a need to notify others within the building as well as people he walked by on the street.

In some implementations example, Bluetooth data can be combined with other sensor data (e.g., imaging data) to get more detailed and accurate risk factors. For example, BT beaconing can be aggregated with other sensor data, e.g., video data, to improve accuracy in determining a real amount of contact that an infected person 109 has had with other people. can be combined with other sensor data (e.g., imaging data) to get more detailed and accurate risk factors. In other words, aggregating BT beaconing with other sensor data can result in added environmental awareness, e.g., determining if people passed by an infected person in an open atrium or in an elevator.

The contact tracing system 102 utilize the aggregated data to look at the environment in which the proximity between a person and an infected person occurred, e.g., by comparing badge/elevator data and Bluetooth data to determine that the interaction occurred in an elevator and not outside. For example, system 102 can include a beacon with its own BT radio such that the BT beacon can associate itself as a contact with a first person A, and then when a second person B arrives, they would become linked to the first person A with the beacon as a shared contact. The BT beacon can be incorporated as a part of a camera with analytics, such that the camera can edit or delete its link to each contact based on their exposure to environment or each other.

In some implementations, audio data can be utilized to enhanced risk score determination. For example, detecting loud talking, sneezing, coughing, or the like in collected audio data from distributed sensors can be utilized to adjust the risk score.

In some implementations, window/door sensors can be utilized to enhance risk score determination. For example, detecting if windows are open/closed or if doors are open/closed can be utilized to determine ventilation, cross-breeze, etc. of a particular area of the property 104.

In some implementations, a baseline risk score can be generated for each sub-area 106a and common area 108 of a property 104. The baseline risk score can depend in part on, for example, air circulation, room size, presence of mitigating factors (e.g., shields, sanitizers, etc.), and the like. For example, a sub-area 106a including a HEPA-filter, may have a lower baseline risk score relative to a sub-area 106b not including a HEPA-filter. In another example, a common area 108 including shields between seating locations may have a lower baseline risk score relative to a common area without the shields.

In some implementations, the exposure evaluation module 124 can determine a risk score based on modes of transmission and other risk factors associated with a particular epidemiological event (e.g., the infection of the infected person 109). In other words, for a particular epidemiological event, different exposures can be associated with a higher or lower risk of infection. For example, a norovirus can be associated with high risk of surface transmission, e.g., an infected person 109 touching a surface in a common space. In another example, influenza can be associated with high risk of droplet transmission, e.g., where proximity/duration of contact between people can be associated with an increased risk score.

In some implementations, the exposure evaluation module 124 requests sensor data 112 for a date/time period, e.g., for a particular date and for a time frame. In one example, the requested aggregated sensor data 112 may include a range of dates for which the infected person 109 was last located at the property 104 based on the provided information related to the infected person 109. In another example, the requested sensor data 112 may include a range of dates including a present date, e.g., a current date and a number of preceding days.

In some implementations, exposure evaluation module 124 can determine a reliability of the collected sensor data 112 as evidence of a potential exposure event. A measure of confidence can be applied to the collected sensor data 112. In other words, a confidence score can be applied to the sensor data 112 collected from a sensor 110 or aggregated sensor data 112 from multiple sensors 110 that reflects a confidence that an exposure event 113 occurred. Confidence scores can include, for example, a rating on a scale, e.g., 1-10, or a rating of high/medium/low.

In some implementations, an assigned confidence score can depend in part on a type of sensor data 112 used to determine the confidence score. The confidence score can be weighted based in part on a type of sensor 110 that has generated the sensor data 112, e.g., imaging data from a camera 123a can be weighed more heavily than badge scanner data. In one example, if a badge scan data indicates a exposure event 113 occurred but the camera indicates no exposure event 113, the confidence score assigned to the exposure event 113 may be low.

The exposure evaluation module 124 can provide confirmation of the exposure event 113 as output to the alert generation module 126. The alert generation module 126 can receive the confirmation of the exposure event 113, e.g., including a confidence score, risk score, and prediction of likelihood of exposure, and generate a exposure response as output.

In some implementations, the exposure evaluation module 124 can aggregate multiple risk scores that a person A can accumulate through a set of potential exposures to infected people resulting in multiple exposure events. When the person's aggregated risk score reaches a threshold score, the contact tracing system 102 can provide a notification to the person including information of the aggregated risk score.

The alert generation module 126 receives a risk score for an exposure event for a person A as input and generates an alert 132. An alert can be provided to a user device 130. Alert 132 can be an SMS/text, robocall, email, pop-up window on user device 130, or a notification provided in an application environment of an application 128 on user device 130, e.g., a contact tracing application. The alert 132 can be provided to the potentially exposed person A and/or to a 3^{rd} party contact tracing entity 117, e.g., a public health authority. In some implementations, the alert 132 can be provided to a management company, business owner, or other interested party, where personal details related to person A may be removed from the alert.

In some implementations, alert 132 can include information relation to mitigation steps responsive to the particular infection to which the person A has potentially been exposed. Mitigation steps can include, for example, self-monitoring, methods to avoid future spread, etc. The alert 132 may include symptoms of the epidemiological event to assist the person to monitor if they themselves have contracted the infection. The alert 132 may include testing locations, and other public health resources (e.g., links to government informative webpages).

In some implementations, contact tracing system 102 can utilize video analytics on collected sensor data, e.g., via biometrics, to perform real-time health monitoring of a person at property 104. A real-time risk assessment of a potentially infected person 109 can include detecting symptoms of illness, e.g., coughing, fever, etc. In response, the system 102 can provide notifications to persons, e.g., person A, who are in contact with the potentially infected person 109.

In some implementations, the contact tracing system 102 can process sensor data 112, e.g., using video analytics, to determine if a person located at property 104 is participating in risky behaviors, e.g., not wearing a mask, coughing, touching their face, etc., that increase their potential risk of contracting an infection. The system can provide a real-time notification to the person including mitigation steps, e.g., a reminder to practice social distancing.

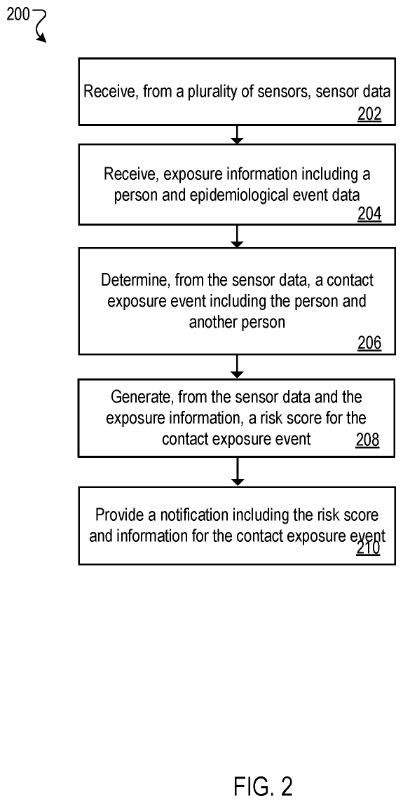

FIG. 2 is a flow diagram of an example process 200 of a targeted response system. The system receives sensor data from multiple sensors (202). Sensor data 112 can be collected by a sensor data collection module 122 from multiple different sensors, for example, imaging data from camera system 125, access data from keycard or badge sensors, connectivity data from wireless receivers, and the like. Sensor data 112 can include environmental factors, for example, temperature, humidity, status of HVAC systems, air purification, etc.

The system receives exposure information including a person and an epidemiological event data (204). An infected person 109 can self-report an illness to the contact tracing system 102, e.g., through application 128, and/or a 3^{rd} party contact tracing entity 117, e.g., public health organization, can provide the information related to the illness to the contact tracing system 102. In some implementations, receiving exposure information including a person and an epidemiological event data includes receiving an indication that the person was infected with an illness at a particular time. For example, the exposure information can include date/time of the infected person's presence at the property 104 and the illness experienced, e.g., a virus/bacteria, onset of symptoms, severity, etc. The exposure information can additionally include a list of locations visited by the infected person 109, people with whom the infected person interacted, and the like. In some implementations, receiving an indication that the person was infected with an illness at a particular time includes receiving an indication that the person that was infected with an illness visited a particular subarea in a property at a particular time. For example, the infected person 109 may indicate that when they were ill they visited common area 108 and sub-areas 106a and 106b.

The system determines, from the sensor data, a contact exposure event including the person and another person (206). A contact exposure event is an occurrence of a potential exposure between an infected person and another person, resulting in the other person having a degree of exposure to the same infection affecting the infected person. The degree of exposure experienced by the other person is determined based on the amount of direct and/or indirect contact the other person experienced with the infected person during the contact exposure event and is affected by various factors, e.g., proximity/duration of exposure, modes of transmission of infection, mitigating factors, environmental factors, and the like.

Exposure evaluation module 124 can determine, from aggregated sensor data 112, direct and/or indirect contact that the infected person had with other people present at the property 104. Direct and/or indirect contact can be determined by the exposure evaluation module 124 by analyzing different streams of sensor data 112 from different sensors 110, e.g., video data from camera system 125 and access data from badge/keycard access points. For example, determining direct/indirect contact can include identifying, access control data (e.g., badge swipes) into a particular sub-area 106a from the infected person 109 and another person A that occur within a threshold amount of time of each other, and identifying, using facial recognition and human models, the infected person in video data collected by the camera system and further determining, using facial recognition and human models, that the other person overlaps spatially/temporally with the infected person in the sub-area 106a.

In some implementations, determining, from the sensor data, a contact exposure event including the person and another person includes identifying, based on the sensor data, presence of the person at a property, identifying, based on the sensor data, presence of the another person at the property, and determining the contact exposure event based on the presence of the person at the property and the presence of the another person at the property. For example, the exposure evaluation module 124 may determine from elevator badge data that both Person A, who was indicated as being sick, and Person B were simultaneously in the same elevator and, based on that determination, determine a contact exposure event.

In some implementations, identifying, based on the sensor data, presence of the person at a property includes identifying the person at the property based on imaging data from a camera system installed in the property. For example, the exposure evaluation module 124 may determine from facial recognition on video captured by the camera 123a that the Person A is in the sub-area 103a.

The system generates, from the sensor data and the exposure information, a risk score for the contact exposure event (208). Exposure evaluation module 124 can determine a risk score for the contact exposure event including the infected person and the other person by aggregating multiple risk factors extracted from the aggregated sensor data 112 including, for example, proximity/duration of exposure, mitigating factors, environmental factors, and modes of transmission of the infection. The generated risk score for the contact exposure event can reflect a risk of transmission of the infection between the infected person and the other person A. In other words, a likelihood that the other person A will contract the infection.

In some implementations, generating, from the sensor data and the exposure information, a risk score for the contact exposure event includes determining a duration and a proximity of the person and the another person at a property based on the sensor data and generating the risk score for the contact exposure event based on the duration and the proximity of the person and the another person at the property based on the sensor data. For example, the exposure evaluation module 124 may determine that the Person A and the Person B were within three feet of each other for two minutes and, based on that determination, determine a risk score that indicates a 50% probability that Person B will contract an illness from Person A.

In some implementations, generating the risk score for the contact exposure event based on sensor data from a heating, ventilation, and air conditioning system installed in a property. For example, the exposure evaluation module 124 may determine that ventilation for an elevator was high and based on that determination, determine a risk score of 25% for the contact exposure event between Persons A and B. In another example, the exposure evaluation module 124 may determine that ventilation for an elevator was low and based on that determination, determine a risk score of 75% for the contact exposure event between Persons A and B.

In some implementations, generating, from the sensor data and the exposure information, a risk score for the contact exposure event includes determining, based on the sensor data, that the person was sneezing or coughing at a property and generating the risk score for the contact exposure event based on that the person was sneezing or coughing at a property.

For example, the exposure evaluation module 124 may determine that ventilation for an elevator was high and based on that determination, determine a risk score of 25% for the contact exposure event between Persons A and B. In another example, the exposure evaluation module 124 may determine that ventilation for an elevator was low and based on that determination, determine a risk score of 75% for the contact exposure event between Persons A and B.

In some implementations, the risk score can be an aggregation of multiple contact exposure events, e.g., the person A came into direct/indirect contact with multiple infected people. The risk score can reflect multiple exposures to a same infection, e.g., each exposure can result in an increased risk score indicating a higher probability that the person A will contract the infection.

The system provides a notification including the risk score and information for the contact exposure event (210). Alert generation module 126 can provide an alert 132 to one or more end-users of the contact tracing system 102 including, for example, potentially exposed person A, 3^{rd} party contract tracing entity 117, business owner, property management, and the like.

In some implementations, the system 102 can detect risky behaviors (e.g., coughing, lack of social distancing) in real-time and send alerts 132 to involved parties to enforce mitigation strategies.

In some implementations, the system 102 can aggregate data collected by other anonymous contact tracing systems by inserting the system 102 itself as a BLE link. For example, person A walks in the elevator and “exposes” a local beacon via the inbuilt contact tracing API running on both the phone and the elevator. When a second person enters the elevator later, the elevator can decide whether to now “expose” person B or not, via the inbuilt contact tracing on their phone. In this scenario, the elevator can either present as the same “person” (e.g., who has had contact with person A) or as a new one (e.g., who will only have contact with person B, in order to potentially link them to a future person C). The API and the inbuilt contact tracing system can handles the risk score determination as described above, but the elevator is able to create a link that otherwise would not have existed to represent this transmission list (i.e., phone A and B were never in proximity with each other).

In some implementations, the system 102 can determine, from the aggregated sensor data 110, an increased risk of infection spread, e.g., a person is observed sneezing or coughing in imaging data collected by the surveillance system. An increased risk of infection can be determined from 3rd party contact tracing entity reports, e.g., that cases of a particular epidemiological event are increasing. In response to determining that there is an increased risk of infection and/or infection spread, the system 102 can determine one or more mitigating steps to reduce spread of the infection. In one example, the system 102 can generate control signals to alter a state of an HVAC system for the property 104, e.g., to change heat, humidity, ventilation states, etc. In another example, the system 102 may determine from video captured by a camera that two people are in a room and that one person sneezed and, in response, instruct the HVAC system to turn on or increase a speed of a fan that ventilates the room.

In some implementations, a location of the increased risk can be identified from the aggregated sensor data 110, e.g., geolocation data, video data, audio data of coughing, etc., and targeted mitigation steps can be taken in response. In one example, a volume of a sneeze picked up at an audio receiver can be utilized by the system 102 to determine where the sneeze occurred, and further to determine an affected volume(s) of air and affected systems. The system 102 can further determine appropriate new settings (e.g., temperature, humidity, airflow rate, filtration level, duration, etc.,) in response to the localized increased risk, and execute the mitigation steps based on the new settings.

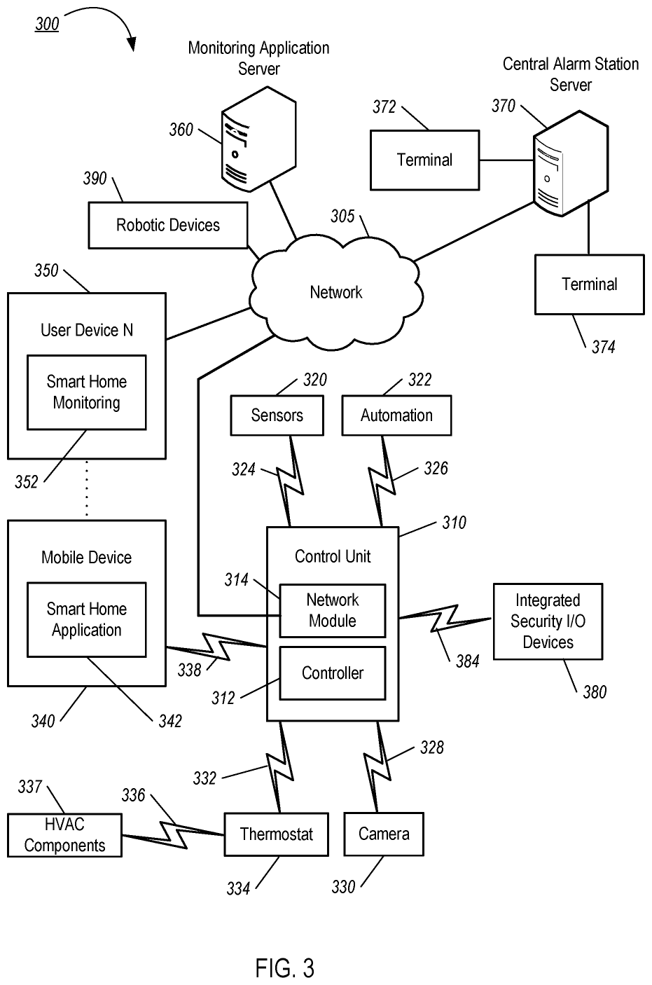

FIG. 3 is a diagram illustrating an example of a home monitoring system 300. The monitoring system 300 includes a network 305, a control unit 310, one or more user devices 340 and 350, a monitoring server 360, and a central alarm station server 370. In some examples, the network 305 facilitates communications between the control unit 310, the one or more user devices 340 and 350, the monitoring server 360, and the central alarm station server 370.

The network 305 is configured to enable exchange of electronic communications between devices connected to the network 305. For example, the network 305 may be configured to enable exchange of electronic communications between the control unit 310, the one or more user devices 340 and 350, the monitoring server 360, and the central alarm station server 370. The network 305 may include, for example, one or more of the Internet, Wide Area Networks (WANs), Local Area Networks (LANs), analog or digital wired and wireless telephone networks (e.g., a public switched telephone network (PSTN), Integrated Services Digital Network (ISDN), a cellular network, and Digital Subscriber Line (DSL)), radio, television, cable, satellite, or any other delivery or tunneling mechanism for carrying data. Network 305 may include multiple networks or subnetworks, each of which may include, for example, a wired or wireless data pathway. The network 305 may include a circuit-switched network, a packet-switched data network, or any other network able to carry electronic communications (e.g., data or voice communications). For example, the network 305 may include networks based on the Internet protocol (IP), asynchronous transfer mode (ATM), the PSTN, packet-switched networks based on IP, X.25, or Frame Relay, or other comparable technologies and may support voice using, for example, VoIP, or other comparable protocols used for voice communications. The network 305 may include one or more networks that include wireless data channels and wireless voice channels. The network 305 may be a wireless network, a broadband network, or a combination of networks including a wireless network and a broadband network.

The control unit 310 includes a controller 312 and a network module 314. The controller 312 is configured to control a control unit monitoring system (e.g., a control unit system) that includes the control unit 310. In some examples, the controller 312 may include a processor or other control circuitry configured to execute instructions of a program that controls operation of a control unit system. In these examples, the controller 312 may be configured to receive input from sensors, flow meters, or other devices included in the control unit system and control operations of devices included in the household (e.g., speakers, lights, doors, etc.). For example, the controller 312 may be configured to control operation of the network module 314 included in the control unit 310.

The network module 314 is a communication device configured to exchange communications over the network 305. The network module 314 may be a wireless communication module configured to exchange wireless communications over the network 305. For example, the network module 314 may be a wireless communication device configured to exchange communications over a wireless data channel and a wireless voice channel. In this example, the network module 314 may transmit alarm data over a wireless data channel and establish a two-way voice communication session over a wireless voice channel. The wireless communication device may include one or more of a LTE module, a GSM module, a radio modem, cellular transmission module, or any type of module configured to exchange communications in one of the following formats: LTE, GSM or GPRS, CDMA, EDGE or EGPRS, EV-DO or EVDO, UMTS, or IP.

The network module 314 also may be a wired communication module configured to exchange communications over the network 305 using a wired connection. For instance, the network module 314 may be a modem, a network interface card, or another type of network interface device. The network module 314 may be an Ethernet network card configured to enable the control unit 310 to communicate over a local area network and/or the Internet. The network module 314 also may be a voice band modem configured to enable the alarm panel to communicate over the telephone lines of Plain Old Telephone Systems (POTS).

The control unit system that includes the control unit 310 includes one or more sensors. For example, the monitoring system may include multiple sensors 320. The sensors 320 may include a lock sensor, a contact sensor, a motion sensor, or any other type of sensor included in a control unit system. The sensors 320 also may include an environmental sensor, such as a temperature sensor, a water sensor, a rain sensor, a wind sensor, a light sensor, a smoke detector, a carbon monoxide detector, an air quality sensor, etc. The sensors 320 further may include a health monitoring sensor, such as a prescription bottle sensor that monitors taking of prescriptions, a blood pressure sensor, a blood sugar sensor, a bed mat configured to sense presence of liquid (e.g., bodily fluids) on the bed mat, etc. In some examples, the health-monitoring sensor can be a wearable sensor that attaches to a user in the home. The health-monitoring sensor can collect various health data, including pulse, heart rate, respiration rate, sugar or glucose level, bodily temperature, or motion data.

The sensors 320 can also include a radio-frequency identification (RFID) sensor that identifies a particular article that includes a pre-assigned RFID tag.

The control unit 310 communicates with the home automation controls 322 and a camera 330 to perform monitoring. The home automation controls 322 are connected to one or more devices that enable automation of actions in the home. For instance, the home automation controls 322 may be connected to one or more lighting systems and may be configured to control operation of the one or more lighting systems. In addition, the home automation controls 322 may be connected to one or more electronic locks at the home and may be configured to control operation of the one or more electronic locks (e.g., control Z-Wave locks using wireless communications in the Z-Wave protocol). Further, the home automation controls 322 may be connected to one or more appliances at the home and may be configured to control operation of the one or more appliances. The home automation controls 322 may include multiple modules that are each specific to the type of device being controlled in an automated manner. The home automation controls 322 may control the one or more devices based on commands received from the control unit 310. For instance, the home automation controls 322 may cause a lighting system to illuminate an area to provide a better image of the area when captured by a camera 330.

The camera 330 may be a video/photographic camera or other type of optical sensing device configured to capture images. For instance, the camera 330 may be configured to capture images of an area within a building or home monitored by the control unit 310. The camera 330 may be configured to capture single, static images of the area and also video images of the area in which multiple images of the area are captured at a relatively high frequency (e.g., thirty images per second). The camera 330 may be controlled based on commands received from the control unit 310.

The camera 330 may be triggered by several different types of techniques. For instance, a Passive Infra-Red (PIR) motion sensor may be built into the camera 330 and used to trigger the camera 330 to capture one or more images when motion is detected. The camera 330 also may include a microwave motion sensor built into the camera and used to trigger the camera 330 to capture one or more images when motion is detected. The camera 330 may have a “normally open” or “normally closed” digital input that can trigger capture of one or more images when external sensors (e.g., the sensors 320, PIR, door/window, etc.) detect motion or other events. In some implementations, the camera 330 receives a command to capture an image when external devices detect motion or another potential alarm event. The camera 330 may receive the command from the controller 312 or directly from one of the sensors 320.

In some examples, the camera 330 triggers integrated or external illuminators (e.g., Infra-Red, Z-wave controlled “white” lights, lights controlled by the home automation controls 322, etc.) to improve image quality when the scene is dark. An integrated or separate light sensor may be used to determine if illumination is desired and may result in increased image quality.

The camera 330 may be programmed with any combination of time/day schedules, system “arming state”, or other variables to determine whether images should be captured or not when triggers occur. The camera 330 may enter a low-power mode when not capturing images. In this case, the camera 330 may wake periodically to check for inbound messages from the controller 312. The camera 330 may be powered by internal, replaceable batteries if located remotely from the control unit 310. The camera 330 may employ a small solar cell to recharge the battery when light is available. Alternatively, the camera 330 may be powered by the controller's 312 power supply if the camera 330 is co-located with the controller 312.

In some implementations, the camera 330 communicates directly with the monitoring server 360 over the Internet. In these implementations, image data captured by the camera 330 does not pass through the control unit 310 and the camera 330 receives commands related to operation from the monitoring server 360.

The system 300 also includes thermostat 334 to perform dynamic environmental control at the home. The thermostat 334 is configured to monitor temperature and/or energy consumption of an HVAC system associated with the thermostat 334, and is further configured to provide control of environmental (e.g., temperature) settings. In some implementations, the thermostat 334 can additionally or alternatively receive data relating to activity at a home and/or environmental data at a home, e.g., at various locations indoors and outdoors at the home. The thermostat 334 can directly measure energy consumption of the HVAC system associated with the thermostat, or can estimate energy consumption of the HVAC system associated with the thermostat 334, for example, based on detected usage of one or more components of the HVAC system associated with the thermostat 334. The thermostat 334 can communicate temperature and/or energy monitoring information to or from the control unit 310 and can control the environmental (e.g., temperature) settings based on commands received from the control unit 310.

In some implementations, the thermostat 334 is a dynamically programmable thermostat and can be integrated with the control unit 310. For example, the dynamically programmable thermostat 334 can include the control unit 310, e.g., as an internal component to the dynamically programmable thermostat 334. In addition, the control unit 310 can be a gateway device that communicates with the dynamically programmable thermostat 334. In some implementations, the thermostat 334 is controlled via one or more home automation controls 322.

A module 337 is connected to one or more components of an HVAC system associated with a home, and is configured to control operation of the one or more components of the HVAC system. In some implementations, the module 337 is also configured to monitor energy consumption of the HVAC system components, for example, by directly measuring the energy consumption of the HVAC system components or by estimating the energy usage of the one or more HVAC system components based on detecting usage of components of the HVAC system. The module 337 can communicate energy monitoring information and the state of the HVAC system components to the thermostat 334 and can control the one or more components of the HVAC system based on commands received from the thermostat 334.

In some examples, the system 300 further includes one or more robotic devices 390. The robotic devices 390 may be any type of robots that are capable of moving and taking actions that assist in home monitoring. For example, the robotic devices 390 may include drones that are capable of moving throughout a home based on automated control technology and/or user input control provided by a user. In this example, the drones may be able to fly, roll, walk, or otherwise move about the home. The drones may include helicopter type devices (e.g., quad copters), rolling helicopter type devices (e.g., roller copter devices that can fly and roll along the ground, walls, or ceiling) and land vehicle type devices (e.g., automated cars that drive around a home). In some cases, the robotic devices 390 may be devices that are intended for other purposes and merely associated with the system 300 for use in appropriate circumstances. For instance, a robotic vacuum cleaner device may be associated with the monitoring system 300 as one of the robotic devices 390 and may be controlled to take action responsive to monitoring system events.

In some examples, the robotic devices 390 automatically navigate within a home. In these examples, the robotic devices 390 include sensors and control processors that guide movement of the robotic devices 390 within the home. For instance, the robotic devices 390 may navigate within the home using one or more cameras, one or more proximity sensors, one or more gyroscopes, one or more accelerometers, one or more magnetometers, a global positioning system (GPS) unit, an altimeter, one or more sonar or laser sensors, and/or any other types of sensors that aid in navigation about a space. The robotic devices 390 may include control processors that process output from the various sensors and control the robotic devices 390 to move along a path that reaches the desired destination and avoids obstacles. In this regard, the control processors detect walls or other obstacles in the home and guide movement of the robotic devices 390 in a manner that avoids the walls and other obstacles.

In addition, the robotic devices 390 may store data that describes attributes of the home. For instance, the robotic devices 390 may store a floorplan and/or a three-dimensional model of the home that enables the robotic devices 390 to navigate the home. During initial configuration, the robotic devices 390 may receive the data describing attributes of the home, determine a frame of reference to the data (e.g., a home or reference location in the home), and navigate the home based on the frame of reference and the data describing attributes of the home. Further, initial configuration of the robotic devices 390 also may include learning of one or more navigation patterns in which a user provides input to control the robotic devices 390 to perform a specific navigation action (e.g., fly to an upstairs bedroom and spin around while capturing video and then return to a home charging base). In this regard, the robotic devices 390 may learn and store the navigation patterns such that the robotic devices 390 may automatically repeat the specific navigation actions upon a later request.

In some examples, the robotic devices 390 may include data capture and recording devices. In these examples, the robotic devices 390 may include one or more cameras, one or more motion sensors, one or more microphones, one or more biometric data collection tools, one or more temperature sensors, one or more humidity sensors, one or more air flow sensors, and/or any other types of sensors that may be useful in capturing monitoring data related to the home and users in the home. The one or more biometric data collection tools may be configured to collect biometric samples of a person in the home with or without contact of the person. For instance, the biometric data collection tools may include a fingerprint scanner, a hair sample collection tool, a skin cell collection tool, and/or any other tool that allows the robotic devices 390 to take and store a biometric sample that can be used to identify the person (e.g., a biometric sample with DNA that can be used for DNA testing).

In some implementations, the robotic devices 390 may include output devices. In these implementations, the robotic devices 390 may include one or more displays, one or more speakers, and/or any type of output devices that allow the robotic devices 390 to communicate information to a nearby user.

The robotic devices 390 also may include a communication module that enables the robotic devices 390 to communicate with the control unit 310, each other, and/or other devices. The communication module may be a wireless communication module that allows the robotic devices 390 to communicate wirelessly. For instance, the communication module may be a Wi-Fi module that enables the robotic devices 390 to communicate over a local wireless network at the home. The communication module further may be a 900 MHz wireless communication module that enables the robotic devices 390 to communicate directly with the control unit 310. Other types of short-range wireless communication protocols, such as Bluetooth, Bluetooth LE, Z-wave, Zigbee, etc., may be used to allow the robotic devices 390 to communicate with other devices in the home. In some implementations, the robotic devices 390 may communicate with each other or with other devices of the system 300 through the network 305.

The robotic devices 390 further may include processor and storage capabilities. The robotic devices 390 may include any suitable processing devices that enable the robotic devices 390 to operate applications and perform the actions described throughout this disclosure. In addition, the robotic devices 390 may include solid-state electronic storage that enables the robotic devices 390 to store applications, configuration data, collected sensor data, and/or any other type of information available to the robotic devices 390.

The robotic devices 390 are associated with one or more charging stations. The charging stations may be located at predefined home base or reference locations in the home. The robotic devices 390 may be configured to navigate to the charging stations after completion of tasks needed to be performed for the monitoring system 300. For instance, after completion of a monitoring operation or upon instruction by the control unit 310, the robotic devices 390 may be configured to automatically fly to and land on one of the charging stations. In this regard, the robotic devices 390 may automatically maintain a fully charged battery in a state in which the robotic devices 390 are ready for use by the monitoring system 300.

The charging stations may be contact based charging stations and/or wireless charging stations. For contact based charging stations, the robotic devices 390 may have readily accessible points of contact that the robotic devices 390 are capable of positioning and mating with a corresponding contact on the charging station. For instance, a helicopter type robotic device may have an electronic contact on a portion of its landing gear that rests on and mates with an electronic pad of a charging station when the helicopter type robotic device lands on the charging station. The electronic contact on the robotic device may include a cover that opens to expose the electronic contact when the robotic device is charging and closes to cover and insulate the electronic contact when the robotic device is in operation.

For wireless charging stations, the robotic devices 390 may charge through a wireless exchange of power. In these cases, the robotic devices 390 need only locate themselves closely enough to the wireless charging stations for the wireless exchange of power to occur. In this regard, the positioning needed to land at a predefined home base or reference location in the home may be less precise than with a contact based charging station. Based on the robotic devices 390 landing at a wireless charging station, the wireless charging station outputs a wireless signal that the robotic devices 390 receive and convert to a power signal that charges a battery maintained on the robotic devices 390.

In some implementations, each of the robotic devices 390 has a corresponding and assigned charging station such that the number of robotic devices 390 equals the number of charging stations. In these implementations, the robotic devices 390 always navigate to the specific charging station assigned to that robotic device. For instance, a first robotic device may always use a first charging station and a second robotic device may always use a second charging station.

In some examples, the robotic devices 390 may share charging stations. For instance, the robotic devices 390 may use one or more community charging stations that are capable of charging multiple robotic devices 390. The community charging station may be configured to charge multiple robotic devices 390 in parallel. The community charging station may be configured to charge multiple robotic devices 390 in serial such that the multiple robotic devices 390 take turns charging and, when fully charged, return to a predefined home base or reference location in the home that is not associated with a charger. The number of community charging stations may be less than the number of robotic devices 390.

In addition, the charging stations may not be assigned to specific robotic devices 390 and may be capable of charging any of the robotic devices 390. In this regard, the robotic devices 390 may use any suitable, unoccupied charging station when not in use. For instance, when one of the robotic devices 390 has completed an operation or is in need of battery charge, the control unit 310 references a stored table of the occupancy status of each charging station and instructs the robotic device to navigate to the nearest charging station that is unoccupied.

The system 300 further includes one or more integrated security devices 380. The one or more integrated security devices may include any type of device used to provide alerts based on received sensor data. For instance, the one or more control units 310 may provide one or more alerts to the one or more integrated security input/output devices 380. Additionally, the one or more control units 310 may receive one or more sensor data from the sensors 320 and determine whether to provide an alert to the one or more integrated security input/output devices 380.

The sensors 320, the home automation controls 322, the camera 330, the thermostat 334, and the integrated security devices 380 may communicate with the controller 312 over communication links 324, 326, 328, 332, 338, and 384. The communication links 324, 326, 328, 332, 338, and 384 may be a wired or wireless data pathway configured to transmit signals from the sensors 320, the home automation controls 322, the camera 330, the thermostat 334, and the integrated security devices 380 to the controller 312. The sensors 320, the home automation controls 322, the camera 330, the thermostat 334, and the integrated security devices 380 may continuously transmit sensed values to the controller 312, periodically transmit sensed values to the controller 312, or transmit sensed values to the controller 312 in response to a change in a sensed value.

The communication links 324, 326, 328, 332, 338, and 384 may include a local network. The sensors 320, the home automation controls 322, the camera 330, the thermostat 334, and the integrated security devices 380, and the controller 312 may exchange data and commands over the local network. The local network may include 802.11 “Wi-Fi” wireless Ethernet (e.g., using low-power Wi-Fi chipsets), Z-Wave, Zigbee, Bluetooth, “Homeplug” or other “Powerline” networks that operate over AC wiring, and a Category 5 (CATS) or Category 6 (CAT6) wired Ethernet network. The local network may be a mesh network constructed based on the devices connected to the mesh network.

The monitoring server 360 is an electronic device configured to provide monitoring services by exchanging electronic communications with the control unit 310, the one or more user devices 340 and 350, and the central alarm station server 370 over the network 305. For example, the monitoring server 360 may be configured to monitor events generated by the control unit 310. In this example, the monitoring server 360 may exchange electronic communications with the network module 314 included in the control unit 310 to receive information regarding events detected by the control unit 310. The monitoring server 360 also may receive information regarding events from the one or more user devices 340 and 350.

In some examples, the monitoring server 360 may route alert data received from the network module 314 or the one or more user devices 340 and 350 to the central alarm station server 370. For example, the monitoring server 360 may transmit the alert data to the central alarm station server 370 over the network 305.

The monitoring server 360 may store sensor and image data received from the monitoring system and perform analysis of sensor and image data received from the monitoring system. Based on the analysis, the monitoring server 360 may communicate with and control aspects of the control unit 310 or the one or more user devices 340 and 350.

The monitoring server 360 may provide various monitoring services to the system 300. For example, the monitoring server 360 may analyze the sensor, image, and other data to determine an activity pattern of a resident of the home monitored by the system 300. In some implementations, the monitoring server 360 may analyze the data for alarm conditions or may determine and perform actions at the home by issuing commands to one or more of the controls 322, possibly through the control unit 310.

The monitoring server 360 can be configured to provide information (e.g., activity patterns) related to one or more residents of the home monitored by the system 300. For example, one or more of the sensors 320, the home automation controls 322, the camera 330, the thermostat 334, and the integrated security devices 380 can collect data related to a resident including location information (e.g., if the resident is home or is not home) and provide location information to the thermostat 334.

The central alarm station server 370 is an electronic device configured to provide alarm monitoring service by exchanging communications with the control unit 310, the one or more user devices 340 and 350, and the monitoring server 360 over the network 305. For example, the central alarm station server 370 may be configured to monitor alerting events generated by the control unit 310. In this example, the central alarm station server 370 may exchange communications with the network module 314 included in the control unit 310 to receive information regarding alerting events detected by the control unit 310. The central alarm station server 370 also may receive information regarding alerting events from the one or more user devices 340 and 350 and/or the monitoring server 360.

The central alarm station server 370 is connected to multiple terminals 372 and 374. The terminals 372 and 374 may be used by operators to process alerting events. For example, the central alarm station server 370 may route alerting data to the terminals 372 and 374 to enable an operator to process the alerting data. The terminals 372 and 374 may include general-purpose computers (e.g., desktop personal computers, workstations, or laptop computers) that are configured to receive alerting data from a server in the central alarm station server 370 and render a display of information based on the alerting data. For instance, the controller 312 may control the network module 314 to transmit, to the central alarm station server 370, alerting data indicating that a sensor 320 detected motion from a motion sensor via the sensors 320. The central alarm station server 370 may receive the alerting data and route the alerting data to the terminal 372 for processing by an operator associated with the terminal 372. The terminal 372 may render a display to the operator that includes information associated with the alerting event (e.g., the lock sensor data, the motion sensor data, the contact sensor data, etc.) and the operator may handle the alerting event based on the displayed information.

In some implementations, the terminals 372 and 374 may be mobile devices or devices designed for a specific function. Although FIG. 3 illustrates two terminals for brevity, actual implementations may include more (and, perhaps, many more) terminals.

The one or more authorized user devices 340 and 350 are devices that host and display user interfaces. For instance, the user device 340 is a mobile device that hosts or runs one or more native applications (e.g., the home monitoring application 342). The user device 340 may be a cellular phone or a non-cellular locally networked device with a display. The user device 340 may include a cell phone, a smart phone, a tablet PC, a personal digital assistant (“PDA”), or any other portable device configured to communicate over a network and display information. For example, implementations may also include Blackberry-type devices (e.g., as provided by Research in Motion), electronic organizers, iPhone-type devices (e.g., as provided by Apple), iPod devices (e.g., as provided by Apple) or other portable music players, other communication devices, and handheld or portable electronic devices for gaming, communications, and/or data organization. The user device 340 may perform functions unrelated to the monitoring system, such as placing personal telephone calls, playing music, playing video, displaying pictures, browsing the Internet, maintaining an electronic calendar, etc.

The user device 340 includes a home monitoring application 352. The home monitoring application 342 refers to a software/firmware program running on the corresponding mobile device that enables the user interface and features described throughout. The user device 340 may load or install the home monitoring application 342 based on data received over a network or data received from local media. The home monitoring application 342 runs on mobile devices platforms, such as iPhone, iPod touch, Blackberry, Google Android, Windows Mobile, etc. The home monitoring application 342 enables the user device 340 to receive and process image and sensor data from the monitoring system.

The user device 340 may be a general-purpose computer (e.g., a desktop personal computer, a workstation, or a laptop computer) that is configured to communicate with the monitoring server 360 and/or the control unit 310 over the network 305. The user device 340 may be configured to display a smart home user interface for the home monitoring application 352 that is generated by the user device 340 or generated by the monitoring server 360. For example, the user device 340 may be configured to display a user interface (e.g., a web page) provided by the monitoring server 360 that enables a user to perceive images captured by the camera 330 and/or reports related to the monitoring system. Although FIG. 3 illustrates two user devices for brevity, actual implementations may include more (and, perhaps, many more) or fewer user devices.

In some implementations, the one or more user devices 340 and 350 communicate with and receive monitoring system data from the control unit 310 using the communication link 338. For instance, the one or more user devices 340 and 350 may communicate with the control unit 310 using various local wireless protocols such as Wi-Fi, Bluetooth, Z-wave, Zigbee, HomePlug (ethernet over power line), or wired protocols such as Ethernet and USB, to connect the one or more user devices 340 and 350 to local security and automation equipment. The one or more user devices 340 and 350 may connect locally to the monitoring system and its sensors and other devices. The local connection may improve the speed of status and control communications because communicating through the network 305 with a remote server (e.g., the monitoring server 360) may be significantly slower.

Although the one or more user devices 340 and 350 are shown as communicating with the control unit 310, the one or more user devices 340 and 350 may communicate directly with the sensors and other devices controlled by the control unit 310. In some implementations, the one or more user devices 340 and 350 replace the control unit 310 and perform the functions of the control unit 310 for local monitoring and long range/offsite communication.

In other implementations, the one or more user devices 340 and 350 receive monitoring system data captured by the control unit 310 through the network 305. The one or more user devices 340, 350 may receive the data from the control unit 310 through the network 305 or the monitoring server 360 may relay data received from the control unit 310 to the one or more user devices 340 and 350 through the network 305. In this regard, the monitoring server 360 may facilitate communication between the one or more user devices 340 and 350 and the monitoring system.

In some implementations, the one or more user devices 340 and 350 may be configured to switch whether the one or more user devices 340 and 350 communicate with the control unit 310 directly (e.g., through link 338) or through the monitoring server 360 (e.g., through network 305) based on a location of the one or more user devices 340 and 350. For instance, when the one or more user devices 340 and 350 are located close to the control unit 310 and in range to communicate directly with the control unit 310, the one or more user devices 340 and 350 use direct communication. When the one or more user devices 340 and 350 are located far from the control unit 310 and not in range to communicate directly with the control unit 310, the one or more user devices 340 and 350 use communication through the monitoring server 360.

Although the one or more user devices 340 and 350 are shown as being connected to the network 305, in some implementations, the one or more user devices 340 and 350 are not connected to the network 305. In these implementations, the one or more user devices 340 and 350 communicate directly with one or more of the monitoring system components and no network (e.g., Internet) connection or reliance on remote servers is needed.

In some implementations, the one or more user devices 340 and 350 are used in conjunction with only local sensors and/or local devices in a house. In these implementations, the system 300 includes the one or more user devices 340 and 350, the sensors 320, the home automation controls 322, the camera 330, and the robotic devices 390. The one or more user devices 340 and 350 receive data directly from the sensors 320, the home automation controls 322, the camera 330, and the robotic devices 390, and sends data directly to the sensors 320, the home automation controls 322, the camera 330, and the robotic devices 390. The one or more user devices 340, 350 provide the appropriate interfaces/processing to provide visual surveillance and reporting.

In other implementations, the system 300 further includes network 305 and the sensors 320, the home automation controls 322, the camera 330, the thermostat 334, and the robotic devices 390, and are configured to communicate sensor and image data to the one or more user devices 340 and 350 over network 305 (e.g., the Internet, cellular network, etc.). In yet another implementation, the sensors 320, the home automation controls 322, the camera 330, the thermostat 334, and the robotic devices 390 (or a component, such as a bridge/router) are intelligent enough to change the communication pathway from a direct local pathway when the one or more user devices 340 and 350 are in close physical proximity to the sensors 320, the home automation controls 322, the camera 330, the thermostat 334, and the robotic devices 390 to a pathway over network 305 when the one or more user devices 340 and 350 are farther from the sensors 320, the home automation controls 322, the camera 330, the thermostat 334, and the robotic devices 390.

In some examples, the system leverages GPS information from the one or more user devices 340 and 350 to determine whether the one or more user devices 340 and 350 are close enough to the sensors 320, the home automation controls 322, the camera 330, the thermostat 334, and the robotic devices 390 to use the direct local pathway or whether the one or more user devices 340 and 350 are far enough from the sensors 320, the home automation controls 322, the camera 330, the thermostat 334, and the robotic devices 390 that the pathway over network 305 is required.

In other examples, the system leverages status communications (e.g., pinging) between the one or more user devices 340 and 350 and the sensors 320, the home automation controls 322, the camera 330, the thermostat 334, and the robotic devices 390 to determine whether communication using the direct local pathway is possible. If communication using the direct local pathway is possible, the one or more user devices 340 and 350 communicate with the sensors 320, the home automation controls 322, the camera 330, the thermostat 334, and the robotic devices 390 using the direct local pathway. If communication using the direct local pathway is not possible, the one or more user devices 340 and 350 communicate with the sensors 320, the home automation controls 322, the camera 330, the thermostat 334, and the robotic devices 390 using the pathway over network 305.

In some implementations, the system 300 provides end users with access to images captured by the camera 330 to aid in decision making. The system 300 may transmit the images captured by the camera 330 over a wireless WAN network to the user devices 340 and 350. Because transmission over a wireless WAN network may be relatively expensive, the system 300 can use several techniques to reduce costs while providing access to significant levels of useful visual information (e.g., compressing data, down-sampling data, sending data only over inexpensive LAN connections, or other techniques).

In some implementations, a state of the monitoring system and other events sensed by the monitoring system may be used to enable/disable video/image recording devices (e.g., the camera 330). In these implementations, the camera 330 may be set to capture images on a periodic basis when the alarm system is armed in an “away” state, but set not to capture images when the alarm system is armed in a “home” state or disarmed. In addition, the camera 330 may be triggered to begin capturing images when the alarm system detects an event, such as an alarm event, a door-opening event for a door that leads to an area within a field of view of the camera 330, or motion in the area within the field of view of the camera 330. In other implementations, the camera 330 may capture images continuously, but the captured images may be stored or transmitted over a network when needed.

The described systems, methods, and techniques may be implemented in digital electronic circuitry, computer hardware, firmware, software, or in combinations of these elements. Apparatus implementing these techniques may include appropriate input and output devices, a computer processor, and a computer program product tangibly embodied in a machine-readable storage device for execution by a programmable processor. A process implementing these techniques may be performed by a programmable processor executing a program of instructions to perform desired functions by operating on input data and generating appropriate output. The techniques may be implemented in one or more computer programs that are executable on a programmable system including at least one programmable processor coupled to receive data and instructions from, and to transmit data and instructions to, a data storage system, at least one input device, and at least one output device.

Each computer program may be implemented in a high-level procedural or object-oriented programming language, or in assembly or machine language if desired; and in any case, the language may be a compiled or interpreted language. Suitable processors include, by way of example, both general and special purpose microprocessors. Generally, a processor will receive instructions and data from a read-only memory and/or a random access memory. Storage devices suitable for tangibly embodying computer program instructions and data include all forms of non-volatile memory, including by way of example semiconductor memory devices, such as Erasable Programmable Read-Only Memory (EPROM), Electrically Erasable Programmable Read-Only Memory (EEPROM), and flash memory devices; magnetic disks such as internal hard disks and removable disks; magneto-optical disks; and Compact Disc Read-Only Memory (CD-ROM). Any of the foregoing may be supplemented by, or incorporated in, specially designed ASICs (application-specific integrated circuits).

It will be understood that various modifications may be made. For example, other useful implementations could be achieved if steps of the disclosed techniques were performed in a different order and/or if components in the disclosed systems were combined in a different manner and/or replaced or supplemented by other components. Accordingly, other implementations are within the scope of the disclosure.

What is claimed is:

1. A computer-implemented method comprising:
receiving sensor data from multiple sensors;
receiving exposure information including a person and an epidemiological event data;
determining, from the sensor data, a contact exposure event including the person and another person;
generating, from the sensor data and the exposure information, a risk score for the contact exposure event; and
providing a notification including the risk score and information for the contact exposure event.

2. The method of claim 1, wherein determining, from the sensor data, a contact exposure event including the person and another person comprises:
identifying, based on the sensor data, presence of the person at a property;
identifying, based on the sensor data, presence of the another person at the property; and
determining the contact exposure event based on the presence of the person at the property and the presence of the another person at the property.

3. The method of claim 2, wherein identifying, based on the sensor data, presence of the person at a property comprises:
identifying the person at the property based on imaging data from a camera system installed in the property.

4. The method of claim 1, wherein generating, from the sensor data and the exposure information, a risk score for the contact exposure event comprises:
determining a duration and a proximity of the person and the another person at a property based on the sensor data; and
generating the risk score for the contact exposure event based on the duration and the proximity of the person and the another person at the property based on the sensor data.

5. The method of claim 1, wherein generating the risk score for the contact exposure event is based on sensor data from a heating, ventilation, and air conditioning system installed in a property.

6. The method of claim 1, wherein generating, from the sensor data and the exposure information, a risk score for the contact exposure event comprises:
determining, based on the sensor data, that the person was sneezing or coughing at a property; and
generating the risk score for the contact exposure event based on that the person was sneezing or coughing at a property.

7. The method of claim 1, wherein receiving exposure information including a person and an epidemiological event data comprises:
receiving an indication that the person was infected with an illness at a particular time.

8. The method of claim 7, wherein receiving an indication that the person was infected with an illness at a particular time comprises:
receiving an indication that the person that was infected with an illness visited a particular subarea in a property at a particular time.

9. A system comprising:
one or more computers and one or more storage devices storing instructions that are operable, when executed by the one or more computers, to cause the one or more computers to perform operations comprising:
receiving sensor data from multiple sensors;
receiving exposure information including a person and an epidemiological event data;
determining, from the sensor data, a contact exposure event including the person and another person;
generating, from the sensor data and the exposure information, a risk score for the contact exposure event; and
providing a notification including the risk score and information for the contact exposure event.

10. The system of claim 9, wherein determining, from the sensor data, a contact exposure event including the person and another person comprises:
identifying, based on the sensor data, presence of the person at a property;
identifying, based on the sensor data, presence of the another person at the property; and
determining the contact exposure event based on the presence of the person at the property and the presence of the another person at the property.

11. The system of claim 10, wherein identifying, based on the sensor data, presence of the person at a property comprises:
identifying the person at the property based on imaging data from a camera system installed in the property.

12. The system of claim 9, wherein generating, from the sensor data and the exposure information, a risk score for the contact exposure event comprises:
determining a duration and a proximity of the person and the another person at a property based on the sensor data; and
generating the risk score for the contact exposure event based on the duration and the proximity of the person and the another person at the property based on the sensor data.

13. The system of claim 9, wherein generating the risk score for the contact exposure event is based on sensor data from a heating, ventilation, and air conditioning system installed in a property.

14. The system of claim 9, wherein generating, from the sensor data and the exposure information, a risk score for the contact exposure event comprises:
determining, based on the sensor data, that the person was sneezing or coughing at a property; and
generating the risk score for the contact exposure event based on that the person was sneezing or coughing at a property.

15. The system of claim 9, wherein receiving exposure information including a person and an epidemiological event data comprises:
receiving an indication that the person was infected with an illness at a particular time.

16. The system of claim 15, wherein receiving an indication that the person was infected with an illness at a particular time comprises:
receiving an indication that the person that was infected with an illness visited a particular subarea in a property at a particular time.

17. A non-transitory computer-readable medium storing software comprising instructions executable by one or more computers which, upon such execution, cause the one or more computers to perform operations comprising:
receiving sensor data from multiple sensors;
receiving exposure information including a person and an epidemiological event data;
determining, from the sensor data, a contact exposure event including the person and another person;
generating, from the sensor data and the exposure information, a risk score for the contact exposure event; and
providing a notification including the risk score and information for the contact exposure event.

18. The medium of claim 17, wherein determining, from the sensor data, a contact exposure event including the person and another person comprises:
identifying, based on the sensor data, presence of the person at a property;
identifying, based on the sensor data, presence of the another person at the property; and
determining the contact exposure event based on the presence of the person at the property and the presence of the another person at the property.

19. The medium of claim 18, wherein identifying, based on the sensor data, presence of the person at a property comprises:
identifying the person at the property based on imaging data from a camera system installed in the property.

20. The medium of claim 17, wherein generating, from the sensor data and the exposure information, a risk score for the contact exposure event comprises:
determining a duration and a proximity of the person and the another person at a property based on the sensor data; and
generating the risk score for the contact exposure event based on the duration and the proximity of the person and the another person at the property based on the sensor data.
